(19) United States
(12) Patent Application Publication
(10) Pub. No.: US 2013/0053425 A1
(43) Pub. Date: Feb. 28, 2013

(54) Method for Lowering Intraocular Pressure Using Gap Junction Blockers

(71) Applicants: Chi-Ho To, Hong Kong (HK); Chi-Wal Do, Hong Kong (HK); Choung-Sing Law, Hong Kong (HK); Yan-Yin Tse, Hong Kong (HK)

(72) Inventors: Chi-Ho To, Hong Kong (HK); Chi-Wal Do, Hong Kong (HK); Choung-Sing Law, Hong Kong (HK); Yan-Yin Tse, Hong Kong (HK)

(21) Appl. No.: 13/221,785

(22) Filed: Aug. 30, 2011

(51) Int. Cl. A61K 31/407; A61P 27/02; A61P 27/06; A61K 31/045

(52) U.S. Cl. 514/411; 514/724

(57) ABSTRACT

A pharmaceutical composition contains a gap junction blocker, and a pharmaceutically acceptable carrier. In addition, a pharmaceutical composition contains a gap junction blocker, a pharmaceutically acceptable carrier, a preservative and a buffer. Also, a method of lowering intraocular pressure includes the step of administrating to a subject a pharmaceutical composition containing a gap junction blocker, and a pharmaceutically acceptable carrier.

### FIELD OF THE INVENTION

The present invention relates to a pharmaceutical composition and a method for lowering intraocular pressure.

### BACKGROUND OF THE INVENTION

Glaucoma is a leading cause of irreversible blindness worldwide. It is commonly associated with elevated intraocular pressure, which leads to damages to retinal ganglion cells, and can progress to blindness if untreated. Intraocular pressure is the measure of the dynamic balance between the secretion (inflow) and drainage (outflow) of the aqueous humour, which is a fluid continuously secreted by the ciliary epithelium of the eye. Once secreted, the aqueous humour enters the eye from the anterior chamber through the pupil and exits mainly through the trabecular meshwork, or to a lesser extent, through the uveoscleral outflow pathway. The trabecular meshwork drains the aqueous humour via the Schlemm's canal into the scleral plexuses and general blood circulation. Elevated intraocular pressure results from insufficient drainage of aqueous humour from the eye, and is a major risk factor for glaucoma.

There are two major types of glaucoma: open-angle glaucoma and closed-angle glaucoma. Open-angle glaucoma accounts for approximately 90% of all glaucoma cases in the US. It is caused by reduced drainage of aqueous humour due to blockages in the trabecular meshwork, resulting in elevated intraocular pressure. Closed-angle glaucoma only accounts for ˜10% of glaucoma cases in the US but up to 50% in other nations. It is characterized by contact between the iris and the trabecular meshwork, which obstructs the outflow of aqueous humour, leading to an increase in intraocular pressure. In about 50% of closed-angle glaucoma cases, prolonged contact between the iris and the trabecular meshwork results in the formation of synechia, which causes permanent obstruction of aqueous outflow.

As retinal ganglion cell death is irreversible, there is presently no cure for glaucoma, nor is there any treatment modality for the rescue or regeneration of the damaged retinal ganglion cells. Current treatments for glaucoma include surgeries and medications which aim to delay the onset and progression of glaucoma-associated vision loss by lowering intraocular pressure.

Surgeries routinely performed to delay the progression of glaucoma generally involve introducing incisions on the iris, sclera or the trabecular meshwork, thereby encouraging aqueous humour outflow and reducing intraocular pressure. Common surgeries for treating glaucoma include laser trabeculoplasty, irodotomy, canaloplasty, trabeculectomy, implantation of glaucoma drainage implants, and non-penetrating deep sclerectomy. Albeit effective in some cases, these surgeries are usually only considered when first line medications fail to control the conditions of the disease. This is because glaucoma surgeries are invasive and often involve complicated procedures that can only be performed by an eye surgeon, meaning that patients undergoing the surgeries have to be admitted to a hospital, causing considerable discomfort and inconvenience.

Although anti-glaucoma medications are less invasive than surgeries, they do suffer from certain drawbacks. There are several classes of anti-glaucoma medications available at present, each functioning via different mechanisms, which are briefly described here. Prostaglandin analogs and miotic agents function by increasing the outflow of aqueous humour from the eye; non-selective beta-adrenergic receptor antagonists, sympathomimetics, and carbonic anhydrase inhibitors function by reducing the formation or secretion of aqueous humour from the ciliary epithelium; and alpha2-adrenergic agonists function via a dual mechanism of decreasing aqueous humour formation as well as encouraging aqueous humour outflow. Physostigmine and cannabinoid have also been shown to lower intraocular pressure, although the precise mechanisms by which they work are not well understood.

One drawback of existing anti-glaucoma medications is that their efficacies are reported to reduce over time as the body develops drug resistance. However, a bigger drawback is that each class of medication is associated with certain side effects. Prostaglandin analogs are reported to induce redness, stinging, itching, and burning sensations in the eye, as well as causing blurred vision; miotic agents are known to cause dim vision, especially during night time or in dark areas; while patients prescribed with non-selective beta-adrenergic receptor antagonists, carbonic anhydrase inhibitors and alpha2-adrenergic agonists have reported more significant side effects, including amongst others, hypotension, fatigue, depression, reduced pulse rate, as well as stomach, kidney, and memory problems. The use of cannabinoid to treat glaucoma needless mention continues to be controversial due to its hallucinogenic and addictive properties that have long been documented.

A further major drawback associated with existing anti-glaucoma medications is that the efficacy of individual medication varies significantly between patients. This is largely due to the mechanism of action of these medications not being well understood, such that a medication that is effective on one patient might not produce the same effect on another patient. This creates a difficult situation for medical workers as there is no universal prescription that can be administered to every patient to ensure that intraocular pressure is kept at a desired level, and there is no way to work out which medication would work best on a specific patient. As a result, a combination of anti-glaucoma medications is often prescribed to a single patient in order to effectively delay the progression of the disease. Since each anti-glaucoma medication is associated with certain side effects, these regimens significantly reduce patient compliance.

It is therefore an object of the present invention to provide an active ingredient for use in treating glaucoma where the above-mentioned shortcomings are mitigated, or to at least provide a useful alternative to the trade and general public.

### SUMMARY OF THE INVENTION

This invention relates to a pharmaceutical composition containing a gap junction blocker, and a pharmaceutically acceptable carrier.

There is also provided a pharmaceutical composition containing a gap junction blocker, a pharmaceutically acceptable carrier, a preservative and a buffer.

The invention further relates to a method of lowering intraocular pressure including the step of administrating to a subject a pharmaceutical composition containing a gap junction blocker, and a pharmaceutically acceptable carrier.

Without intending to be limited by theory, it is believed that the inventions herein may provide a useful alternative to existing anti-glaucoma medications, as the inventors of the present invention spent much effort on obtaining crucial insights into the mechanisms behind aqueous humor formation, on which the present invention is based. As a result, there is a clear confirmation and understanding on the mechanism of action via which the present inventions function. The present invention thus offers a distinct advantage over many existing anti-glaucoma treatments and medications, for which the mechanism of action is not well understood, and therefore the efficacy unpredictable. Further, as the present invention functions via a mechanism that is distinct from that of existing anti-glaucoma medications, a synergistic effect is also expected when the present invention is used in conjunction with existing anti-glaucoma medications.

### BRIEF DESCRIPTION OF THE DRAWINGS

FIG. 1 illustrates the effects of topical administration of 1.0% heptanol to rabbit eyes.

FIG. 2 illustrates effects of aqueous addition of 3.5 mM heptanol on FF and PD (n=6). FIG. 2A illustrates the calculated FF (fluid flow) rate in the blood-to-aqueous direction during the course of the experiment. FIG. 2B illustrates PD (potential difference) recorded simultaneously with the measurement of the capillary levels.

### DETAILED DESCRIPTION

Unless specifically noted herein as otherwise, any conditions described herein are at 25° C., 50% relative humidity, and atmospheric pressure. Similarly, unless stated otherwise, all measurements are made in metric units, and all ratios, percentages etc., are by weight of the final composition.

The inventors of the present application realized that the varied efficacies of existing anti-glaucoma medications between individuals are largely due to the fact that the precise mechanisms of action for these medications are unknown, owing to the fact that the regulation of aqueous humour formation are still not well-understood. In view of this, the inventors carried out extensive research on the formation of aqueous humour and its transport into and out of the eye, which provided basis for the current invention.

A few decades ago, Na⁺/HCO₃⁻ transport was initially proposed to be the major ion transport mechanisms across the ciliary epithelium, resulting in the secretion of aqueous humour. Later studies however, failed to detect any net transport of Na⁺ across the ciliary epithelium, which casted doubt on this prevailing theory. In fact, until recently, only net Cl⁻ transport could be detected across the ciliary epithelium using radioactive tracers. HCO₃⁻ ion was shown to only indirectly modulate the net Cl⁻ transport across the epithelium. Thus, contrary to the direction of many other researchers, the inventors of the present application postulated that it is Cl⁻, not Na⁺/HCO₃⁻ transport, that provides the crucial driving force for aqueous humour transport across the ciliary epithelium and therefore focused their efforts on studying the transport of Cl⁻ across the ciliary epithelium in order to gain crucial insights into the transport of aqueous humour into the eye.

Electron microscopy revealed the ciliary epithelium to be a dual-layered structure comprising a pigmented epithelium and a non-pigmented epithelium. Numerous transporters and channels line the plasma membranes of the pigmented and non-pigmented epithelium to facilitate transport of solutes across the ciliary epithelium. These transporters, including the Na⁺/H⁺ and Cl⁻/HCO₃⁻ antiporters and the Na⁺—K⁺-2Cl⁻ cotransporter, are believed to be present in the pigmented epithelium for the uptake of ions from the blood plasma into the pigmented epithelial cells. Cl⁻ channels are also found to be present in the non-pigmented epithelium to facilitate the release of ions from the non-pigmented epithelial cells into the aqueous humour. In addition, it was later revealed that protein channels known as the gap junctions are present in between the pigmented and non-pigmented epithelium. Without intending to be limited by theory, the inventors believe that gap junctions provide a crucial conduit for the transport of solutes and fluid between the pigmented and non-pigmented cells, and further postulated that, by blocking the gap junctions, transport of solutes from the pigmented to non-pigmented epithelium can be prevented, thereby, reducing the influx of aqueous humour into the posterior chamber.

Structurally, each gap junction is made up of two connexons (or hemichannels) that interact in a tail-to-tail fashion to form a protein channel that transverse two adjacent cell membranes. Each hemichannel is a homo- or hetero-hexamer of the transmembrane protein connexin. It is widely accepted that a family of structurally-related connexin proteins exist in humans, each differing slightly structurally and functionally, and are present at different locations within the body. Previous studies showed that the connexin Cx43 is the major component of gap junctions present between cardiac myocytes. While this was known for cardiac tissue, investigation of gap junctions in the eye was lacking. However, Cx43 has now also emerged to be the major component constituting the gap junctions between the pigmented and non-pigmented epithelium.

Amongst many others, carbenoxolone, halothane, lindane, octanol, and heptanol are all known gap junction blockers used in cardiotherapy. Heptanol in particular is commonly used in cardio electrophysiology experiments to block gap junctions between myocytes. Without intending to be limited by theory, based on the hypothesis that the gap junctions between cardiac myocytes are similar to those present between the pigmented and non-pigmented epithelium, the inventors proceeded to investigate the effect of heptanol on solutes and fluid transport across the ciliary epithelium.

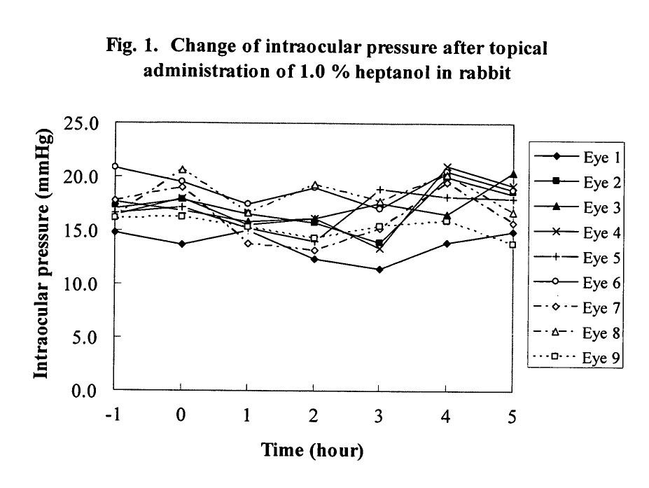

The Inventors set out to test the effectiveness of heptanol in lowering intraocular pressure (IOP) in rabbit in vivo but encountered major problems, as topical administration of 1.0% heptanol to the rabbit eyes produced inconclusive results (FIG. 1).

They then conducted in vitro studies and showed that heptanol reduces short-circuit current by ˜80% across the ciliary epithelium in rabbit, ox and pig, and that the inhibition of short-circuit current is largely mediated by reduction in net Cl⁻ secretion.

Although the inventors successfully demonstrated that heptanol inhibits transport of Ci across the ciliary epithelium, for a long time, there was no direct evidence to show heptanol has any effect on fluid flow in and out of the eye. The reasons being that firstly, no information was available at the time to teach the preparation an intact ciliary epithelium, which is required for fluid flow measurement, and secondly, even when an intact ciliary epithelium was used, there was no device available that would enable measurement of fluid flow across the ciliary epithelium. In view of this, the inventors invested a great deal of effort into developing a modified Ussing-type chamber, which enables fluid flow (FF) and potential difference across the ciliary epithelium to be measured simultaneously.

Fundamentally, the Ussing-type chamber includes two hemi-chambers, between which an excised preparation of ciliary epithelium is mounted. The first hemi-chamber is connected to a bubbling reservoir containing physiological Ringer's solution with a continuous supply of oxygen, and the second, opposite hemi-chamber is connected to a graduated capillary and maintained as a water-tight compartment. The graduated capillary can be used to monitor the change in fluid volume inside the second hemi-chamber throughout the experiment, which is then calculated as FF rate. The cavity of the chamber is designed to hold the complete annulus of the ciliary epithelium in order to maximize the volume of fluid secreted by the ciliary epithelium. The inventors also developed various techniques for the preparation of intact ciliary epitheliums.

Following the development of the modified Ussing-type chamber and the techniques for preparing intact ciliary epithelium, the inventors spent a long time optimizing the experimental conditions before any success was seen, as initial trials run on ox and pig ciliary epithelium indicated that FF was unstable and declined gradually over time. After much experimentation, the inventors finally realized that the problem was caused by the bathing temperature within the chamber, which was being held at 37° C., and eventually found that a lower temperature of around 25° C. was required to maintain a viable preparation, and that it was only when they conducted the experiments at a lower bathing temperature of 25° C. could the preparation be stabilised for a few hours to enable FF measurements. At a later stage, the inventors further incorporated electrodes into the FF chamber to enable both electrical parameters and FF to be monitored simultaneously.

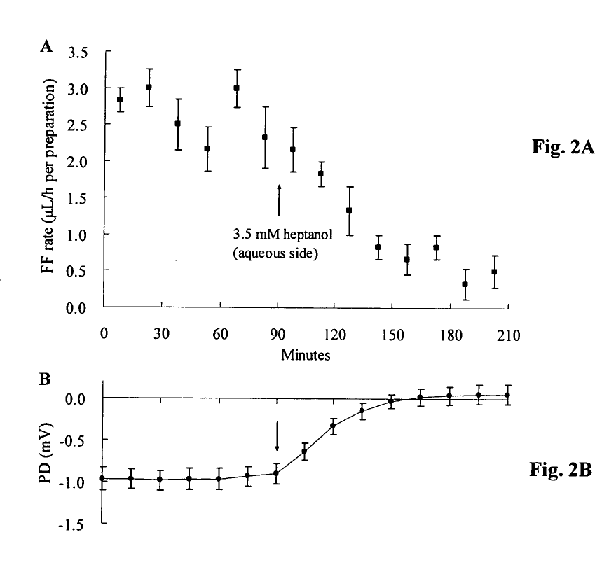

Using the Ussing-type chamber and the optimized protocol described above, the inventors not only successfully showed that fluid flow across the ciliary epithelium is indeed associated with Cl⁻ transport, but also that heptanol inhibits fluid flow by up to 78% in porcine ciliary epithelium (FIG. 2). In other words, the inventor's results provided, for the first time, direct evidence that heptanol is effective in lowering intraocular pressure and could be used as a treatment for glaucoma.

FIG. 2. Effects of aqueous addition of 3.5 mM heptanol on FF and PD (n=6). (A) The calculated FF rate in the blood-to-aqueous direction during the course of the experiment. (B) PD recorded simultaneously with the measurement of the capillary levels.

According to an embodiment of the present invention, there is provided a pharmaceutical composition which includes a gap junction blocker, and a pharmaceutically acceptable carrier. As used here, the term “gap junction blocker” includes the pharmaceutically-acceptable derivatives thereof, such as salts, precursors, etc that are known in the art. In an embodiment herein, the gap junction blocker is selected from a group consisting of heptanol, octanol, anadamide, fenamate, retinoic acid, oleamide, spermine, aminosulphates, halothane, enflurane, isoflurane, propofol, thiopental, glycyrrhetinic acid, quinine, 2-aminoethoxydiphenyl borate, pharmaceutically acceptable derivative of the above compounds, and a combination thereof; or heptanol, octanol, anadamide, fenamate, retinoic acid, oleamide, spermine, aminosulphates, halothane, enflurance, isoflurane, propofol, thiopental, glycyrrhetinic acid, quinine, 2-aminoethoxydiphenyl borate, and a combination thereof; or heptanol, a pharmaceutically acceptable derivative thereof and a combination thereof; or heptanol. In an embodiment herein, the pharmaceutically acceptable derivative of heptanol is selected from a group consisting of 1-heptanol, 2-heptanol, 3-heptanol, 4-heptanol and a combination thereof. A pharmaceutically acceptable derivative of fenamate is selected from a group consisting of meclofenamic acid, niflumic acid, flufenamic acid and a combination thereof. An example of a pharmaceutically acceptable derivative of glycyrrhetinic acid is carbenoxolone. A pharmaceutically acceptable derivative of quinine is selected from a group consisting of quinidine, mefloquine, and a combination thereof.

In an embodiment of the present invention, commercially prepared heptanol can be used to prepare the pharmaceutical composition. Commercially prepared heptanol is widely available from chemical companies such as Sigma-Aldrich, St. Louis, Mo., USA (http://www.sigmaaldrich.com/united-states.html) in a variety of forms and purities. Heptanol can be used at any concentration ranging from about 0.005% to about 20%, or from about 0.05% to about 10%, or from about 0.1% to about 7% by weight of the final composition.

Pharmaceutically acceptable carriers of the pharmaceutical composition include sterile purified water, a sodium chloride solution, a cellulose suspension, a cellulose solution and a combination thereof. If present, the concentration of sodium chloride is generally from about 0.5% to about 0.9% by weight of the final composition.

In an embodiment of the present invention, the pharmaceutical composition also includes a solubilizing agent, such as a 2-hydroxypropyl-β-cyclodextrin (HPβCD) solution, as it is believed that the addition of HPβCD increases the solubility of heptanol in water. The concentration of HPβCD typically ranges from about 1% to about 20%, or from about 5% to about 20% by weight of the final composition.

Heptanol is the first gap junction blocker that has been shown to inhibit fluid flow across the ciliary epithelium. Since this mechanism of action is very different from those of existing anti-glaucoma actives, it is conceivable that the use of heptanol in conjunction with existing anti-glaucoma actives will produce a synergistic effect. Accordingly, in an embodiment of the present invention, the pharmaceutical composition may further include an additional anti-glaucoma active, such as a prostaglandin analogue, a non-selective beta-adrenergic receptor antagonist, an alpha2-adrenergic agonist, a carbonic anhydrase inhibitor, a cholinergic agent, a miotic agent, a sympathomimetic, a physostigmine, cannabinoid, and a combination thereof. The prostaglandin analogue may be, for example about 0.001%-0.01% travaprost, about 0.01-0.03% bimatoprost, about 0.001-0.01% latanoprost and a combination thereof. The non-selective beta-adrenergic receptor antagonist may be, for example, about 0.1-1% timolol maleate, about 0.25-0.5% betaxolol HCl, about 0.25-0.5% levobunolol HCl ophthalmic solution, USP, about 0.1-1% metipranolol, about 0.25-0.5% timolol hemihydrate, about 0.25-0.5% timolol maleate ophthalmic gel forming solution and a combination thereof. The alpha2-adrenergic agonist may be about 0.5-1% apraclonidine HCl, about 0.1-0.15% brimonidine tartrate and a combination thereof. The carbonic anhydrase inhibitor may be about 0.1-5% brinzolamide ophthalmic suspension, about 1-5% dorzolamide HCl, acetazolamide and a combination thereof. The miotic may be about 1-8% pilocarpine HCl, about 0.75-3% of carbachol, about 1-10% of pilocarpine HCl gel, about 0.5-6% pilocarpine HCl ophthalmic solution USP, and a combination thereof.

In an embodiment herein, the pharmaceutical composition is formulated as an eye drop which may further include a preservative, such as benzalkonium chloride, sodium borate, boric acid and a combination thereof. The pH of the composition is typically adjusted to a pH value of about 7.5 using hydrochloric acid and/or sodium hydroxide and maintained using a buffer such as sodium phosphate, sodium citrate and a combination thereof.

The pharmaceutical composition according to an embodiment of the present invention can be manufactured by mixing together the gap junction blocker, HPβCD, and sterile purified water, before adding sodium chloride and benzalkonium chloride to the solution, and adjusting the pH value to about 7.5 using a buffering agent and diluted hydrochloric acid and/or sodium hydroxide.

### Example 1

| | |
|---|---|
| Heptanol | 1% w/v |
| HPβCD | 20% w/v |
| Sodium dihydrogen phosphate | 0.3% w/v |
| Disodium hydrogen phosphate | 0.15% w/v |
| Sodium chloride | 0.5% w/v |
| Benzalkonium chloride | 0.005% w/v |
| Sodium hydroxide or hydrochloric | Suitable amount to adjust pH to 7.5 |
| acid | |
| Sterile purified water | Suitable amount to adjust to a final |
| | volume of 100 ml |

The pharmaceutical composition according to the present invention can be used to lower intraocular pressure through administration to a subject, where the subject is selected from the group consisting of a human, a dog, a cat, a horse, a rabbit, a pig, or a bovine. In an embodiment herein the bovine is an ox or a cow. In an embodiment herein, the subject is first diagnosed with elevated intraocular pressure or a disease associated with elevated intraocular pressure, such as glaucoma and ocular hypertension. The pharmaceutical composition is then administered to the subject.

The pharmaceutical composition can also be administered via any methods described herein, and especially as an eye drop, to a subject to prevent a specific condition, such as pressure-induced death of retinal ganglion cells. Thus, the pharmaceutical composition herein may be used as an eye drop, either alone or in combination with other ingredients.

For administration, the pharmaceutical composition can be formulated as a solution, a gel, an ointment, a suspension, a viscoelastic preparation, and a combination thereof. To formulate the pharmaceutical composition as a gel or a viscoelastic preparation, 0.5-5% of carboxymethylcellulose can be included in the pharmaceutical composition.

The frequency of administrating the pharmaceutical composition can be varied between individuals depending on the severity of their condition and may range from about once per minute, about once per five minutes, about once per fifteen minutes, about hourly, about bihourly, about daily, about weekly, about biweekly, about monthly, about yearly, to about once every two years. The actual frequency may vary greatly from subject to subject, and is often determined by an eye doctor. In an embodiment herein, the pharmaceutical composition is administered to a subject from about once per day to about once per week.

The pharmaceutical composition herein may be administered to a subject by a variety of methods. The administration of the pharmaceutical composition can be performed topically via an ocular route, including the corneal, intra-corneal, subconjuntival, subtenon, episcleral and intra-scleral, intra-cameral routes. Alternatively, the pharmaceutical composition can also be administered via a perioculer, orbital, or extra-ocular route, including intravenous, subcutaneous and intramuscular administration.

The pharmaceutical composition can be administered using an injectable drug delivery system such as intravitreal injection or an implantable drug delivery system such as ocular, peribulbar or retrobulbar implants. In an embodiment herein, these delivery systems are capable of facilitating sustained release of the pharmaceutical composition via a time-controlled drug delivery system that releases the pharmaceutical composition or at least the gap junction blocker over a period of time rather than all at once.

In an embodiment, the pharmaceutical composition is administered via a media selected from, for example, a contact lens, a collagen shield, an ocular insert and a combination thereof, loaded with the pharmaceutical composition. For loading, the media, for example, a contact lens, can be soaked in the pharmaceutical composition from approximately 1 hr to approximately 3 hrs so that the media contains the pharmaceutical composition therein, and/or thereupon. Alternatively, the contact lens can be soaked in the pharmaceutical composition overnight. The pharmaceutical composition is then administered to a subject upon wearing the loaded contact lens.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided or separately or in any suitable subcombination.

Any references or publications cited herein are hereby incorporated by reference herein, in whole or relevant part. However, citation of any reference or publication is not to be construed an admission as to the reference or publication's admissibility or availability as prior art.

What is claimed is:

1. A pharmaceutical composition comprising:
a) a gap junction blocker; and
b) a pharmaceutically acceptable carrier.

2. A pharmaceutical composition according to claim 1 wherein the gap junction blocker is selected from a group consisting of heptanol, octanol, anadamide, fenamate, retinoic acid, oleamide, spermine, aminosulphates, halothane, enflurance, isoflurane, propofol, thiopental, glycyrrhetinic acid, quinine, 2-aminoethoxydiphenyl borate, pharmaceutically acceptable derivatives thereof, and a combination thereof.

3. The pharmaceutical composition according to claim 1 wherein the gap junction blocker is from about 0.005% by weight to about 20% by weight of the final composition.

4. The pharmaceutical composition according to claim 3 wherein the gap junction blocker is from about 0.05% by weight to about 10% by weight of the final composition.

5. The pharmaceutical composition according to claim 3 wherein the gap junction blocker is from about 0.1% by weight to about 7% by weight of the final composition.

6. The pharmaceutical composition according to claim 1 further comprising a solubilizing agent.

7. The pharmaceutical composition according to claim 6 wherein the solubilizing agent is 2-hydroxypropyl-β-cyclodextrin.

8. The pharmaceutical composition according to claim 6 wherein the solubilizing agent is from about 1% by weight to about 20% by weight.

9. The pharmaceutical composition according to claim 1 wherein the pharmaceutically acceptable carrier is selected from a group consisting of sterile purified water, sodium chloride solution, a cellulose suspension, a cellulose solution, and a combination thereof.

10. The pharmaceutical composition according to claim 1 further comprising an additional anti-glaucoma active.

11. The pharmaceutical composition according to claim 10 wherein the additional anti-glaucoma active is selected from a group consisting of a prostaglandin analogue, a non-selective beta-adrenergic receptor antagonist, an alpha2-adrenergic agonist, a carbonic anhydrase inhibitor, a cholinergic agent, a miotic agent, a sympathomimetic, a physostigmine, cannabinoid and a combination thereof.

12. The pharmaceutical composition according to claim 1 in a physical form selected from a group consisting of a solution, a gel, an ointment, a suspension, a viscoelastic preparation, and a combination thereof.

13. The pharmaceutical composition according to claim 12 wherein the physical form is a gel or a viscoelastic preparation and wherein the pharmaceutical composition further comprises carboxymethylcellulose.

14. The pharmaceutical composition according to claim 13 wherein the carboxymethylcellulose is from about 0.5% by weight to 5% by weight of the final composition.

15. The pharmaceutical composition according to claim 1 further comprising a preservative selected from a group consisting of benzalkonium chloride, sodium borate, boric acid and a combination thereof.

16. The pharmaceutical composition according to claim 1 further comprising a buffer selected from the group consisting of sodium phosphate, sodium citrate, and a combination thereof.

17. The use of the pharmaceutical composition according to claim 1 as an eye drop.

18. A pharmaceutical composition comprising:
a) a gap junction blocker thereof;
b) a pharmaceutically acceptable carrier;
c) a preservative; and
d) a buffer.

19. A method for lowering intraocular pressure comprising the step of administrating to a subject a pharmaceutical composition comprising:
a) a gap junction blocker; and
b) a pharmaceutically acceptable carrier.

20. The method according to claim 19 further comprising the step of diagnosing the subject as having ocular hypertension or glaucoma.

21. The method according to claim 19 wherein the subject is selected from a group consisting of a human, a dog, a cat, a horse, a rabbit, a pig, or a bovine.

22. The method according to claim 19 further comprising the step of administrating the pharmaceutical composition at a frequency from about once per minute to about once every two years.

23. The method according to claim 19 wherein the step of administrating the pharmaceutical composition is via an ocular route.

24. The method according to claim 19 wherein the step of administrating the pharmaceutical composition is via an extraocular route.

25. The method according to claim 19 wherein the step of administrating the pharmaceutical composition is via an injectable drug delivery system.

26. The method according to claim 19 wherein the step of administrating the pharmaceutical composition is via an implantable drug delivery system.

27. The method according to claim 19 wherein the step of administrating the pharmaceutical composition is via a time-controlled drug delivery system.

28. The method according to claim 19 wherein the step of administrating the pharmaceutical composition is via a media selected from a group consisting of a contact lens, collagen shield, ocular insert or a combination thereof, and wherein the media comprises the pharmaceutical composition.
